SEQUENCE LISTING (10 sequences)

SEQ ID NO: 1 - DNA, 30 bp, Artificial Sequence
  Description of Artificial Sequence Synthetic Barcode 1 oligonucleotide
cgtttggatccgcatgacattcgccgtaag

SEQ ID NO: 2 - DNA, 30 bp, Artificial Sequence
  Description of Artificial Sequence Synthetic Barcode 2 oligonucleotide
gcaacttacggcgaatgaccgaatcagcct

SEQ ID NO: 3 - DNA, 30 bp, Artificial Sequence
  Description of Artificial Sequence Synthetic Barcode 3 oligonucleotide
gcataggctgattcggttcatgcggatcca

SEQ ID NO: 4 - DNA, 30 bp, Artificial Sequence
  Description of Artificial Sequence Synthetic Barcode 4 oligonucleotide
ttgcaggctgattcggttcatgcggatcca

SEQ ID NO: 5 - DNA, 26 bp, Artificial Sequence
  Description of Artificial Sequence Synthetic Barcode 5 oligonucleotide
tggatccgcatgacattcgccgtaag

SEQ ID NO: 6 - DNA, 26 bp, Artificial Sequence
  Description of Artificial Sequence Synthetic Barcode 6 oligonucleotide
cttacggcgaatgaccgaatcagcct

SEQ ID NO: 7 - DNA, 30 bp, Artificial Sequence
  Description of Artificial Sequence Synthetic Barcode 7 oligonucleotide
atgcaggctgattcggttcatgcggatcca

SEQ ID NO: 8 - DNA, 26 bp, Artificial Sequence
  Description of Artificial Sequence Synthetic Barcode 8 oligonucleotide
cttacggcgaatgaccgaatcagcct

SEQ ID NO: 9 - DNA, 26 bp, Artificial Sequence
  Description of Artificial Sequence Synthetic Barcode 9 oligonucleotide
tggatccgcatgacattcgccgtaag

SEQ ID NO: 10 - DNA, 43 bp, Artificial Sequence
  Description of Artificial Sequence Synthetic A10 oligonucleotide
tagcgggaggacgaugcggaucagccauguuuacgucacuccu

(19) United States
(12) Patent Application Publication
(10) Pub. No.: US 2014/0364596 A1
(43) Pub. Date: Dec. 11, 2014

(54) ARTIFICIAL LIPID-POLYMER-DNA COMPLEX, BIOIMAGING AGENT AND PREPARATION METHOD THEREOF

(71) Applicant: Research & Business Foundation SUNGKYUNKWAN UNIVERSITY, Suwon-si (KR)

(72) Inventors: Soong Ho UM, Seoul (KR); Chang Yoon BAEK, Seoul (KR)

(73) Assignee: Research & Business Foundation SUNGKYUNKWAN UNIVERSITY, Suwon-si (KR)

(21) Appl. No.: 14/466,635

(22) Filed: Aug. 22, 2014

(30) Foreign Application Priority Data
Feb. 24, 2012 (KR) 10-2012-0019306

(51) Int. Cl. A61K 49/00

(52) U.S. Cl. CPC A61K 49/0054
USPC 536/23.1; 428/402

(57) ABSTRACT

An artificial lipid-polymer-DNA complex, an in-vivo imaging agent, a pharmaceutical composition and a method of preparing the same are provided. The artificial lipid bilayer includes a polymer matrix including polylactic-co-glycolic acid disposed within a lipid membrane.

### CROSS-REFERENCE TO RELATED APPLICATIONS

This application is a continuation application of International Application No. PCT/KR2012/007605 filed Sep. 21, 2012, claiming priority based on Korean Patent Application No. 10-2012-019306 filed Feb. 24, 2012, in the Korean Intellectual Property Office, the entire disclosure of all of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on Jul. 24, 2014, is named 042099.0014_SL.txt and is 2,883 bytes in size.

### BACKGROUND

1. Field

The following description disclosure relates to an artificial lipid bilayer, a nucleic acid-based in-vivo imaging agent, a method of preparing the same, and a use thereof.

2. Description of Related Art

With recent explosion of studies regarding methods of diagnosing intractable infectious diseases such as cancer, new diagnostic methods involving the use of various materials are being proposed. Among these methods, analysis methods that use a real-time PCR or DNA microarray using unique physiochemical characteristics of DNA or RNA are in the limelight. However, according to such a diagnostic methodology, a test should be performed several times according to an object of the analysis. Thus, lots of time and costs are needed to treat a large amount of samples. In addition, highly-sensitive and high-cost analysis equipments are necessary to perform the diagnosis. Therefore, it is cumbersome and expensive to use such a diagnostic method. Accordingly, there exists a demand to develop methods that utilize new structures designed based on a nucleic acid having excellent targeting ability, sensitivity, and the like to directly diagnose a target disease in a living organism (U.S. Unexamined Patent Application Publication No. 2007/0048759). However, when such a nucleic acid-based diagnostic structure is administered directly to a living organism, the structure becomes unstable in a blood vessel due to malfunction, degradation and the like and may induce an immune response. Accordingly, despite an excellent diagnostic function, only in vitro application is considered. These issues are applicable not only to nucleic acid-based diagnostic methods, but also in a field of gene therapy that uses a nucleic acid such as siRNA, shRNA, and the like. Accordingly, to effectively make use of such a nucleic acid-based material in a living organism, the non-uniformity in the interaction between a nucleic acid-based material and a living organism must be addressed. Thus, there exists a demand for developing a functional material or a delivery device for a nucleic acid-based material, which can stably protect the nucleic acid-based material inside the body of a living organism and can perform a target-oriented delivery.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, an artificial lipid-polymer-DNA complex includes a polymer matrix including polylactic-co-glycolic acid disposed within a lipid membrane.

The lipid membrane may include 1,2-dioleoyl-sn-Glycero-3-Phosphocholine, 1,2-dioleoyl-sn-glycero-3-[phospho-rac-(1-glycerol)]), and 1,2-dioleoyl-sn-glycero-3-phosphoethanolamin-N-[4-(p-maleimidophenyl)butyramide].

The artificial lipid-polymer-DNA complex may include a spherical shape with a diameter of 50 nm or greater and 10 μm or less.

The artificial lipid-polymer-DNA complex may be configured to deliver in vivo a therapeutic agent disposed in the polymer matrix to a tissue targeted by specificity determined by a nucleic acid aptamer disposed on a surface of the lipid membrane.

In another general aspect, an in-vivo imaging agent includes a nucleic acid in the above-described artificial lipid-polymer-DNA complex.

The artificial lipid-polymer-DNA complex may have a nucleic acid aptamer having cancer cell targeting ability on its surface.

The nucleic acid may be labeled with a fluorescent material.

The nucleic acid may include a nucleic acid-based nano barcode having cancer cell targeting ability.

The nucleic acid-based nano barcode may be combined with at least one linear nucleic acid including one selected from the group consisting of the base sequences of SEQ. ID. NOs: 1 to 9.

The in-vivo imaging agent may include an anticancer agent.

In another general aspect, a pharmaceutical composition for diagnosing or treating a cancer may include the above described in-vivo imaging agent as an active component.

In another general aspect, a method of preparing an in-vivo imaging agent is provided. The method involves: preparing a mixed solution by dissolving 1,2-dioleoyl-sn-glycero-3-phosphocholine1,2-dioleoyl-sn-glycero-3-[phospho-rac-(1-glycerol)], and 1,2-dioleoyl-sn-glycero-3-phosphoethanolamin-N-[4-(p-maleimidophenyl)butyramide] in a concentration ratio of 40:10:50 to 72:18:10 in an organic solvent; preparing a lipid mixture by drying the mixed solution and dissolving the dried product in dichloromethane; preparing a polymer-lipid mixed solution by adding a polylactic-co-glycolic acid (50:50 Poly(D,L-lactide-co-glycolide)) polymer solution dissolved in dichloromethane to the lipid mixture; adding the polymer-lipid mixture to distilled water from which a DNase comprising a nucleic acid is removed, and performing sonication; evaporating dichloromethane from the sonicated polymer-lipid mixture; and homogenizing a particle size using a particle homogenizer (extruder).

The adding of the polymer-lipid mixture to distilled water from which a DNase comprising a nucleic acid is removed may be performed at a temperature of greater than 2° C. and less than 8° C.

The general aspect of the method may further involve binding a nucleic acid aptamer having cancer cell targeting ability to 1,2-dioleoyl-sn-glycero-3-phosphoethanolamin-N-[4-(p-maleimidophenyl)butyramide].

The organic solvent may be chloroform or a chloroform-methanol mixed solution.

Other features and aspects will be apparent from the following detailed description, the drawings, and the claims.

### BRIEF DESCRIPTION OF THE DRAWINGS

FIG. 1A is a schematic diagram illustrating an example of a method of preparing an artificial lipid-polymer-DNA complex.

FIG. 1B is a flowchart illustrating an example of a method of preparing an artificial lipid-polymer-DNA complex.

FIG. 2A is a schematic diagram illustrating an example of a method of preparing an artificial lipid-polymer-DNA complex.

FIG. 2B is a flowchart illustrating an example of a method of preparing an artificial lipid-polymer-DNA complex.

FIG. 3 includes images showing results in which an artificial lipid-polymer-DNA complex observed using a confocal microscope. A scale size is 5 μm.

FIG. 4 shows images showing results in which a polymer matrix from which a lipid membrane surface support is removed is observed using a confocal microscope. A scale size is 5 μm.

FIG. 5 shows images showing results in which sizes of artificial lipid-polymer-DNA complexes are measured using a confocal microscope. A scale size is 1 μm.

FIG. 6 shows images showing results in which sizes of artificial lipid-polymer-DNA complexes are measured using an atomic force microscope. A scale size is 5 μm.

FIG. 7A is a light scattering intensity distribution map illustrating the size of an example of an artificial lipid-polymer-DNA complex according to the present disclosure as measured by a laser scattering method.

FIG. 7B is a scattering number distribution map graph illustrating the size of an example of an artificial lipid-polymer-DNA complex according to the present disclosure as measured by a laser scattering method.

FIG. 8 shows images showing results in which artificial lipid-polymer-DNA complexes including nucleic acids are observed using a confocal microscope. A scale size is 2 μm.

FIG. 9 shows results in which polymer matrixes including a nucleic acid from which a lipid membrane surface support is removed are observed using a confocal microscope. A scale size is 10 μm.

FIG. 10 is a schematic diagram illustrating an example of a method of providing information for diagnosing a disease using an in-vivo imaging agent according to the present disclosure.

FIG. 11 is a schematic diagram illustrating an example of an artificial lipid-polymer-DNA complex according to the present disclosure.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, compositions, materials, agents, devices and structures described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The present disclosure provides an in-vivo imaging agent including a nucleic acid labeled with a fluorescent material within an artificial lipid-polymer-DNA complex that includes a polymer matrix composed of polylactic-co-glycolic acid [50:50 poly(D,L-lactide-co-glycolide); PLGA] inside a surface support formed of a lipid membrane. In addition, the present disclosure provides a pharmaceutical composition for diagnosing or treating cancer, which includes the in-vivo imaging agent as an active component.

However, the technical aspect of the present disclosure is not limited to the above-described examples, and other aspects not described herein will be clearly understood by those of ordinary skill in the art.

According to one aspect of the present disclosure, an artificial lipid-polymer-DNA complex includes a polymer matrix composed of PLGA that is disposed under a surface support formed of a lipid membrane.

According to one example, the lipid membrane is composed of 1,2-dioleoyl-sn-glycero-3-phosphocholine, 1,2-dioleoyl-sn-glycero-3-[phospho-rac-(1-glycerol)], and 1,2-dioleoyl-sn-glycero-3-phosphoethanolamin-N-[4-(p-maleimidophenyl)butyramide].

According to another example, the artificial lipid-polymer-DNA complex may form a spherical shape having a diameter of 50 nm to 10 μm.

Another aspect of the present disclosure provides an in-vivo imaging agent including a nucleic acid labeled with a fluorescent material in the artificial lipid-polymer-DNA complex.

In one embodiment according to the present disclosure, the artificial lipid-polymer-DNA complex includes a nucleic acid aptamer having cancer cell targeting ability on its surface.

In another embodiment according to the present disclosure, the nucleic acid aptamer is an A10 aptamer selectively binding to a prostate cancer cell.

In still another embodiment according to the present disclosure, the nucleic acid includes a nucleic acid-based nano barcode having cancer cell targeting ability.

In still another embodiment according to the present disclosure, the nucleic acid-based nano barcode is combined with at least one linear nucleic acid including one selected from the group consisting of base sequences of SEQ. ID. NOs: 1 to 9.

In yet another embodiment according to the present disclosure, the in-vivo imaging agent includes an anticancer agent.

Still another aspect of the present disclosure provides a pharmaceutical composition for diagnosing or treating a cancer including the in-vivo imaging agent as an active component.

Yet another aspect of the present disclosure provides a method of preparing an in-vivo imaging agent. The method may involve: preparing a mixed solution by dissolving 1,2-dioleoyl-sn-glycero-3-phosphocholine, 1,2-dioleoyl-sn-glycero-3-[phospho-rac-(1-glycerol)]), and 1,2-dioleoyl-sn-glycero-3-phosphoethanolamin-N-[4-(p-maleimidophenyl)butyramide in chloroform or a chloroform-methanol mixed solution to have a concentration ratio of 40:10:50 to 72:18:10, preparing a lipid mixture by drying the mixed solution and dissolving the dried product in dichloromethane, preparing a polymer-lipid mixture by adding a PLGA polymer solution, which has been dissolved in dichloromethane to the lipid mixture, adding the polymer-lipid mixture to distilled water from which a DNase including a nucleic acid is removed at 4° C. and performing sonication, evaporating dichloromethane from the sonicated polymer-lipid mixture, and homogenizing a size of a particle using a particle homogenizer (extruder).

In one embodiment, the method of preparing an in-vivo imaging agent further includes binding a nucleic acid aptamer having cancer cell targeting ability to 1,2-dioleoyl-sn-glycero-3-phosphoethanolamin-N-[4-(p-maleimidophenyl)butyramide].

According to an example of the in-vivo imaging agent of the present disclosure, since the in-vivo imaging agent that includes a nucleic acid-based nano barcode is configured as a lipid membrane that mimics a bio membrane, it can be stably introduced into a cell in a living organism in vivo, and deliver a nucleic acid-based nano barcode with great specificity to a desired position by binding a nucleic acid aptamer having a targeting ability onto a surface of the lipid membrane, thereby considerably increasing target-oriented imaging efficiency. In addition, as a bilayer is formed using a PLGA polymer having biodegradability and biocompatibility, in-vivo toxicity can be considerably decreased, and stability can be further increased. In addition, the in-vivo imaging agent may carry various drugs for treating diseases without limitation as a drug delivery device. Thus, the in-vivo imaging agent has potential to be applied in an in-vivo diagnosis of a disease and in the treatment of a disease.

Herein, various examples of implementing the in-vivo imaging agent are described.

The inventors achieved a functional material or a delivery device for a nucleic acid structure that stably protects the nucleic acid material within the body of a living organism and enables a target-oriented delivery of the nucleic acid material, as well as other therapeutic agents.

The present disclosure provides an in-vivo imaging agent that includes an artificial lipid-polymer-DNA complex. The artificial lipid-polymer-DNA complex may include a polymer matrix composed of PLGA within a surface support formed of a lipid membrane and a nucleic acid labeled with a fluorescent material in the artificial lipid-polymer-DNA complex.

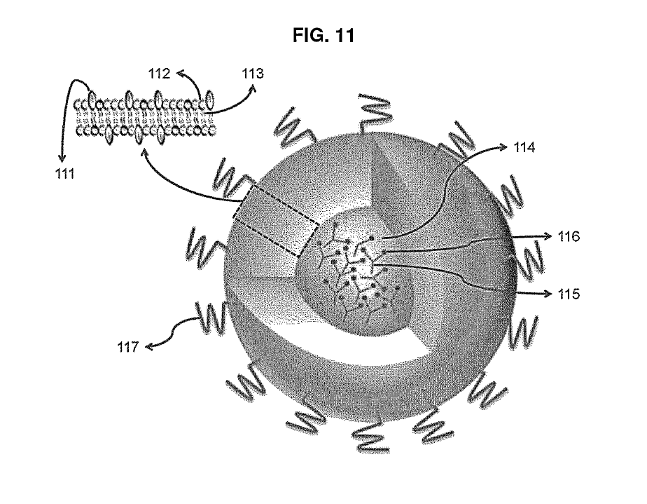

An example of an artificial lipid-polymer-DNA complex is illustrated in FIG. 11. As illustrated in FIG. 11, the artificial lipid-polymer-DNA complex contains three compartments including a lipid outer membrane, an interfacial polymer and a DNA core. A lipid bilayer includes two layers of phospholipid molecules 111. Hydrophilic heads 112 of the phospholipid molecules are aligned along an outer surface of the bilayer that contacts an aqueous medium. Hydrophobic tails 113 of the phophoslipid molecules 111 aggregate inside the bilayer. In this example, the hollow of the bilayer is filed with a polymer matrix 114. The polymer matrix 114 may contain a therapeutic agent or a fluorescent material. In this example, a nucleic acid material 115 labeled with a fluorescent material or intercalated with a dye molecule is disposed within the polymer matrix 114. In addition, a nucleic acid aptamer 117 is provided on the outer surface of the bilayer to enable tissue-specific delivery of the artificial complexes to a target tissue. In addition, the present disclosure provides a method of preparing an in-vivo imaging agent, which includes preparing a mixed solution by dissolving 1,2-dioleoyl-sn-glycero-3-phosphocholine, 1,2-dioleoyl-sn-glycero-3-[phospho-rac-(1-glycerol)]), and 1,2-dioleoyl-sn-glycero-3-phosphoethanolamin-N-[4-(p-maleimidophenyl)butyramide in chloroform or a chloroform-methanol mixed solution to have a concentration ratio of 40:10:50 to 72:18:10, preparing a lipid mixture by drying the mixed solution and dissolving the dried product in dichloromethane, preparing a polymer-lipid mixture by adding a PLGA polymer solution, which has been dissolved in dichloromethane to the lipid mixture, adding the polymer-lipid mixture to distilled water from which a DNase including a nucleic acid is removed at 4° C. and performing sonication, evaporating dichloromethane from the sonicated polymer-lipid mixture, and homogenizing a size of a particle using a particle homogenizer (extruder). Step sequences and/or configuration are not limited, as long as an in-vivo imaging agent including a fluorescent-labeled nucleic acid structure of the present disclosure can be prepared.

The functional material for the nucleic acid structure prepared by the above-described method, that is, an artificial lipid-polymer-DNA complex, may be a bilayer that includes PLGA polymers having biodegradability and biocompatibility. The lipid membrane mimics a bio membrane. Thus, when used a delivery device for a therapeutic agent or imaging material, the artificial lipid membrane reduces toxicity and increase stability of the material inside the body of a living organism. A size of the artificial lipid-polymer-DNA complex is not limited as long as it has a size and a shape that can be injected into a living body, and the artificial lipid-polymer-DNA complex may be a spherical shape having a diameter of 50 nm to 10 μm.

In addition, a maleimide functional group may be present on a surface of the artificial lipid-polymer-DNA complex, and may enable the formation of a covalent bond with a material having a thiol functional group. Thus, molecules having various activities may be bound to the surface of the artificial lipid-polymer-DNA complex. There is no limit to the kind of a bindable molecule. However, the ability of the artificial lipid-polymer-DNA complex to target a specific tissue may be enhanced by binding the nucleic acid aptamer having targeting ability substituted with a thiol functional group. For example, the nucleic acid aptamer that is bound may be an A10 aptamer (SEQ. ID. NO: 10) that selectively binds to a prostate cancer cell.

The inside of the polymer matrix of the artificial lipid-polymer-DNA complex of the present disclosure may be empty, or be filled with various materials. There is no limit to a material that can be included in the polymer matrix. However, according to one example, the polymer matrix includes a nucleic acid and/or anticancer agent labeled with a fluorescent material. Thus, the polymer matrix may be used as an agent for in-vivo imaging and/or treatment. The nucleic acid may include a nucleic acid-based nano barcode having cancer cell targeting ability, or siRNA or shRNA that can be used in cancer treatment. Thus, the polymer matrix may be stained by binding a fluorescent protein to the nucleic acid, or using a fluorescent material such as POPO-3 or rhodamine B. However, there is no limit to a method of labeling the nucleic acid with a fluorescent material.

Therefore, the present disclosure provides a pharmaceutical composition for diagnosing or treating a cancer including the in-vivo imaging agent as an active component.

The pharmaceutical composition of the present disclosure may include a pharmaceutically available carrier. The pharmaceutically available carrier may include saline, polyethyleneglycol, ethanol, vegetable oil, and isopropyl myristate, but the present disclosure is not limited thereto.

The present disclosure also provides a method of treating cancer by administrating a pharmaceutically effective amount of the pharmaceutical composition including the in-vivo imaging agent as an effective component to an individual. The term “individual” used herein refers to a subject having a disease to be treated, and such as, for example, a mammal including as a human, a non-human primate, a mouse, a rat, a dog, a cat, a horse, or a cow. In addition, according to the present disclosure, it is apparent to those of ordinary skill in the art that a range of the pharmaceutically effective amount can be controlled in various ways according to a patient's weight, age, sex, health condition, diet, administration time, administration method, excretion rate, and severity of a disease.

A preferable dosage of the pharmaceutical composition of the present disclosure is dependent on the patient's condition and weight, severity of the disease, drug type, administration route, and duration, and may be suitably selected by those of ordinary skill in the art. However, according to one example, the dosage is approximately 0.001 to 100 mg/kg, or 0.01 to 30 mg/kg per day based on the body weight. The drug may be administered once or several times a day. The in-vivo imaging agent of the present disclosure may be present in an amount of 0.0001 to 10 wt %, or 0.001 to 1 wt % with respect to a total weight of the composition.

The pharmaceutical composition of the present disclosure may be administered to mammals such as rats, mice, livestock, or humans by various routes. There is no limitation to the administration method, and the pharmaceutical composition may be administered by oral, rectal, or intravascular administration, or muscular, subcutaneous, endometrial or intra cerebroventricular injection.

Hereinafter, a number of examples will be described to aid the understanding of the present disclosure. However, the following examples are provided merely so that the present disclosure can be more easily understood, and the scope of the claims is not limited to the following examples.

### EXAMPLES

### Example 1

### Formation of Nucleic Acid-Based Nano Barcode

To prepare a nucleic acid-based nano barcode, a middle support was composed by binding three linear nucleic acids (Barcode 1: 5′-Phos-CGTTTGGATCCGCATGACATTCGCCGTAAG-3′ (SEQ. ID. NO: 1), Barcode 2: 5′-Phos-GCAACTTACGGCGAATGACCGAATCAGCCT-3′ (SEQ. ID. NO: 2), and Barcode 3: 5′-Phos-GCATAGGCTGATTCGGTTCATGCGGATCCA-3′ (SEQ. ID. NO: 3)) to which fluorescent particles did not bind. In addition, a linear nucleic acid to which fluorescent particles did not bind (Barcode 4: 5′-Phos-TTGCAGGCTGATTCGGTTCATGCGGATCCA-3′ (SEQ. ID. NO: 4)) and two linear nucleic acids to which fluorescent particles were bound (Barcode 5: 5′-6FAM-TGGATCCGCATGACATTCGCCGTAAG-3′ (SEQ. ID. NO: 5) and Barcode 6: 5′-Cy5-CTTACGGCGAATGACCGAATCAGCCT-3′ (SEQ. ID. NO: 6)) were bound to be located at one end of the middle support. At the other end of the middle support, one linear nucleic acid to which fluorescent particles is not bound (Barcode 7: 5′-Phos-ATGCAGGCTGATTCGGTTCATGCGGATCCA-3′ (SEQ. ID. NO: 7)) and two linear nucleic acids to which fluorescent particles are bound (Barcode 8: 5′-6FAM-CTTACGGCGAATGACCGAATCAGCCT-3′ (SEQ. ID. NO: 8), and Barcode 9: 5′-Cy5-TGGATCCGCATGACATTCGCCGTAAG-3′(SEQ. ID. NO: 9)) were attached, thereby preparing the nucleic acid-based nano barcode. Complementary hybridization of the base sequences was performed at 95° C. for 2 minutes, 65° C. for 2 minutes, and 60° C. for 5 minutes, and then repeated by decreasing a temperature by −1° C. per 30 seconds at 60° C. The last step of decreasing a temperature by −1° C. per 30 seconds at 60° C. was repeated for 40 cycles. Then, the resulting product was stored at 4° C.

### Example 2

### Formation of Artificial Lipid-Polymer-DNA Complex and In-Vivo Imaging Agent

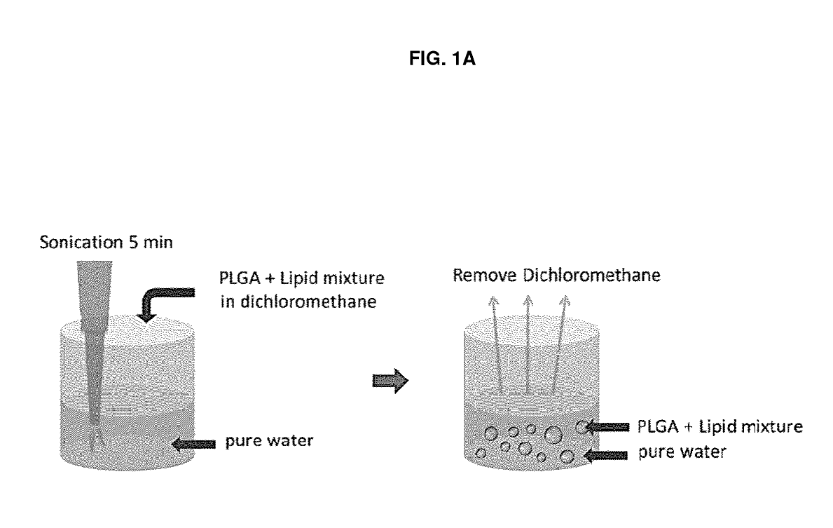

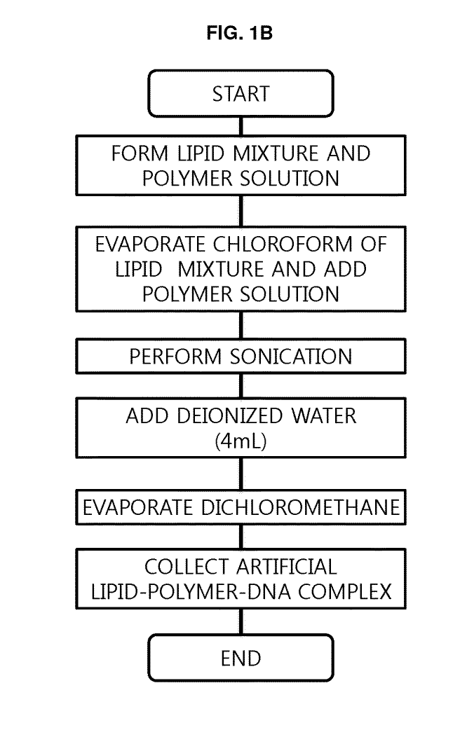

To form a lipid membrane surface support (artificial lipid-polymer-DNA complex) including a polymer matrix as a nucleic acid nano barcode-based in-vivo imaging agent, first, a lipid thin film was formed by mixing lipid components that is, 1,2-dioleoyl-sn-glycero-3-phosphocholine (DOPC, Formula 1), 1,2-dioleoyl-sn-glycero-3-[phospho-rac-(1-glycerol)] (DOPG, Formula 2), and 1,2-Dioleoyl-sn-glycero-3-phosphoethanolamin-N-[4-(p-maleimidophenyl)butyramide] (DSPE-MPB, Formula 3) dissolved in chloroform to have a concentration ratio of 40:10:50 to 72:18:10, adding the resulting solution to a glass bottle, and drying the solution in a rotary evaporator to completely evaporate the chloroform, and then dissolved in 400 μl of dichloromethane again, thereby preparing a lipid mixture. In addition, as the polymer matrix, a PLGA (50:50 poly(D,L-lactide-co-glycolide, Formula 4) having biodegradability and biocompatibility was used. The PLGA was dissolved in dichloromethane to have a concentration of 80 mg/mL, and 100 μl of the above-described solution was mixed with the lipid thin film dissolved in 400 μl of the dichloromethane (a ratio of lipid to polymer=1:18 to 1:25). Afterward, the solution prepared by mixing the lipid and the polymer (polymer-lipid mixture) was added to 4 mL of deionized water at 4° C., and sonicated for 5 minutes, thereby preparing a lipid membrane surface support including the polymer matrix, as illustrated in FIG. 1B. In addition, the solution was stirred for 24 hours using a stirrer to evaporate an organic solvent, that is, dichloromethane, and passed through a 1.0 μm polycarbonate membrane-equipped particle homogenizer (extruder) 21 times to homogenize a size of the lipid membrane surface support including the polymer matrix. The prepared solution was stored at 4° C. A schematic diagram for the test method is shown in FIG. 1A.

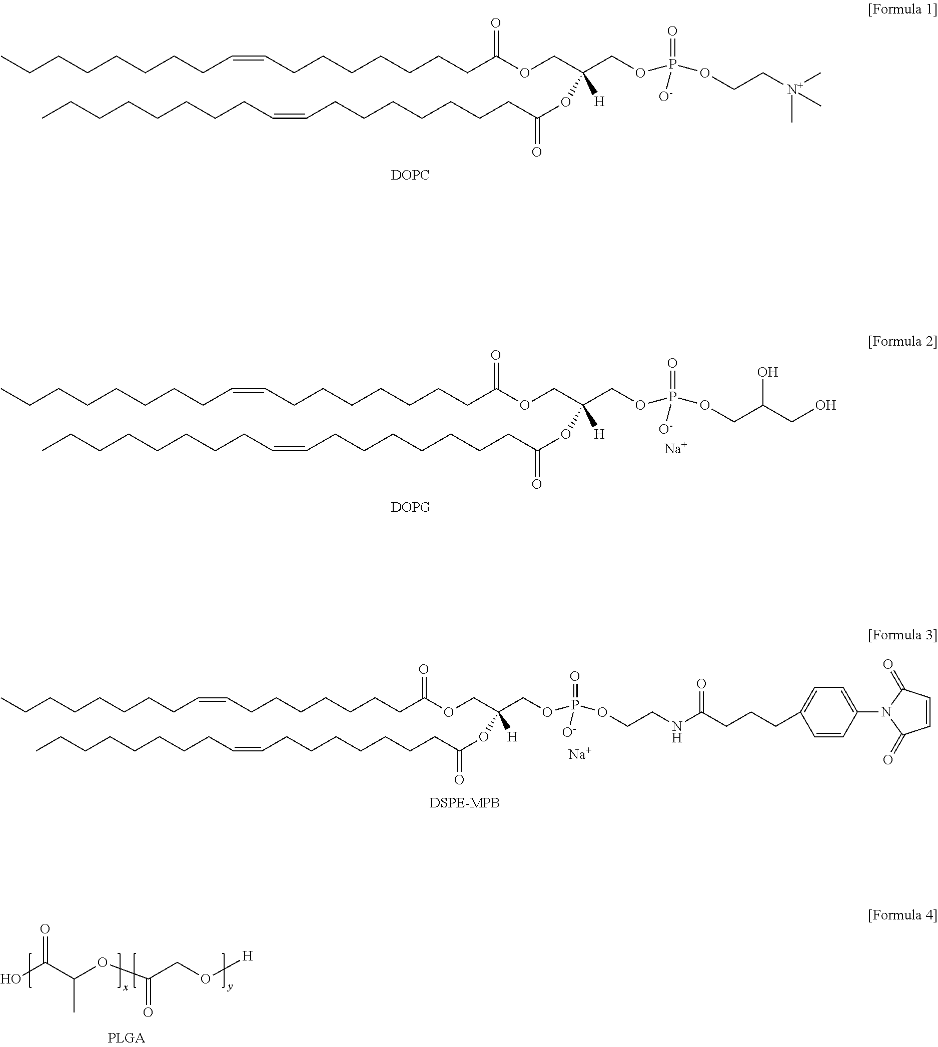

To form an artificial lipid-polymer-DNA complex including a nucleic acid-based nano barcode, that is, an in-vivo imaging agent, instead of the deionized water, distilled water without 4 mL of DNase including 25 μg of the nucleic acid-based barcode prepared by the method of Example 1 was used. In addition, to form a lipid membrane surface support including a polymer matrix having a salmon sperm DNA, instead of the deionized water, distilled water from which 4 mL of a DNase including 25 μg of salmon sperm DNA stained with SYBR Green was removed was used.

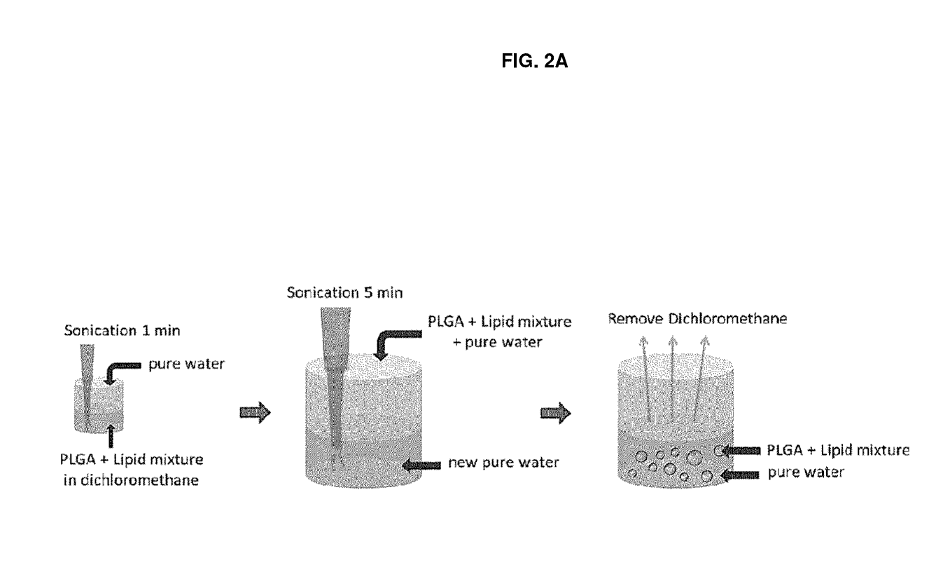

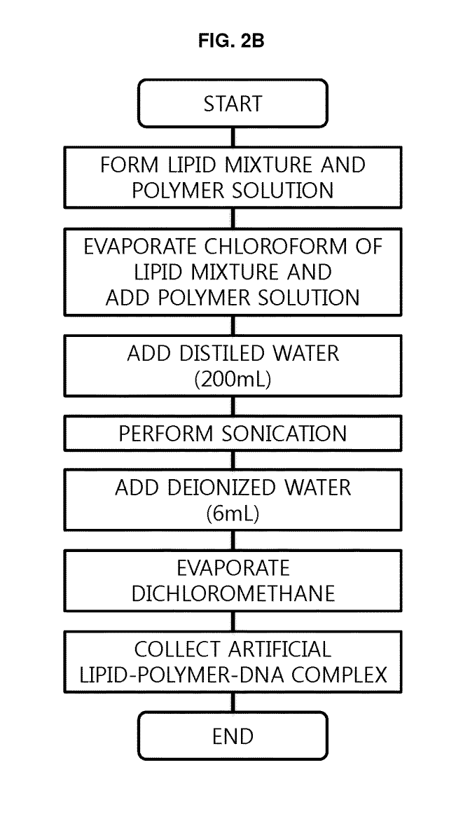

Alternatively, to form an artificial lipid-polymer-DNA complex, a lipid thin film was formed by mixing lipid components, such as DOPC, DOPG, and DSPE-MPB, dissolved in chloroform to have a concentration ratio of 11.7:3:5.53, adding the resulting solution in a glass bottle, and drying the solution in a rotary evaporator to completely evaporate the chloroform, and then dissolved again in 600 μl of dichloromethane. In addition, PLGA was dissolved in dichloromethane to have a concentration of 80 mg/mL, and 400 μl of the resulting solution was mixed with the lipid thin film dissolved in 600 μl of dichloromethane (a ratio of lipid:polymer=1:18 to 1:25). Afterward, 200 μl of deionized water was added to the solution in which the lipid and polymer were mixed by sonication at 4° C. for 1 minute, and the solution was added to 6 mL of deionized water at 4° C. through 5-minute sonication, thereby forming a lipid membrane surface support including a polymer matrix, as illustrated in FIG. 2B. In addition, the solution was stirred using a stirrer for 24 hours to evaporate an organic solvent, such as dichloromethane, and passed through 1.0 μm polycarbonate membrane-equipped particle homogenizer (extruder) 21 times to homogenize a size of the artificial lipid-polymer-DNA complex. The prepared solution was stored at 4° C. A schematic diagram for the test method is shown in FIG. 2A. In the following Example, an artificial lipid-polymer-DNA complex was formed by the both methods for forming an artificial lipid-polymer-DNA complex described above.

### Example 3

### Confirmation of Structure of Artificial Lipid-Polymer-DNA Complex

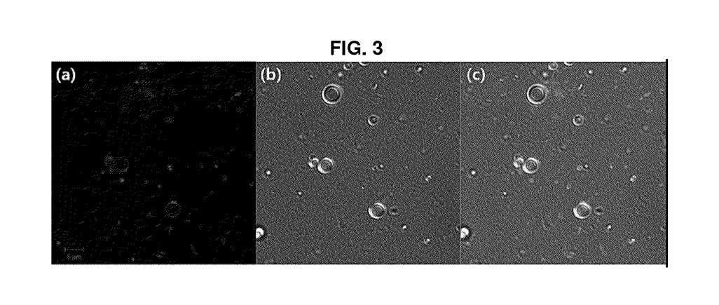

To confirm a structure of the artificial lipid-polymer-DNA complex formed by the method of Example 2, after a fluorescent material specifically binding to the lipid, such as a rhodamine B 1× solution, was diluted 1,000 times and treated to the lipid membrane surface support including the polymer matrix to perform a reaction at room temperature for 1 hour. In addition, the lipid membrane surface support was fixed with a calcium-alginate (Ca-alginate) solution, and observed with a confocal microscope. The results are illustrated in FIG. 3.

Referring to the microscopic image provided in FIG. 3, it was confirmed that the artificial lipid-polymer-DNA complex was formed in a spherical structure by surrounding a surface of the polymer matrix with the rhodamine B-stained lipid membrane.

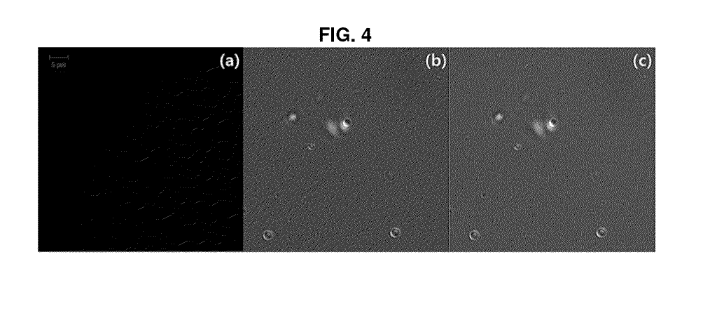

To confirm that the surface of the polymer matrix was surrounded by the lipid membrane, a surfactant specifically removing a lipid, such as a Triton X-100 solution, was diluted 10 times and treated to the rhodamine B-treated solution to perform a reaction at room temperature for 1 hour, and the resulting product was observed using a confocal microscope. The results are illustrated in FIG. 4.

As shown in FIG. 4(b), a spherical structure was optically observed from an optical image, but as shown in FIG. 4(a), rhodamine B was not observed from a fluorescence image. According to the results, it was confirmed that the surface of the polymer matrix was successfully surrounded with the lipid membrane.

### Example 4

### Measurement of Size of Artificial Lipid-Polymer-DNA Complex

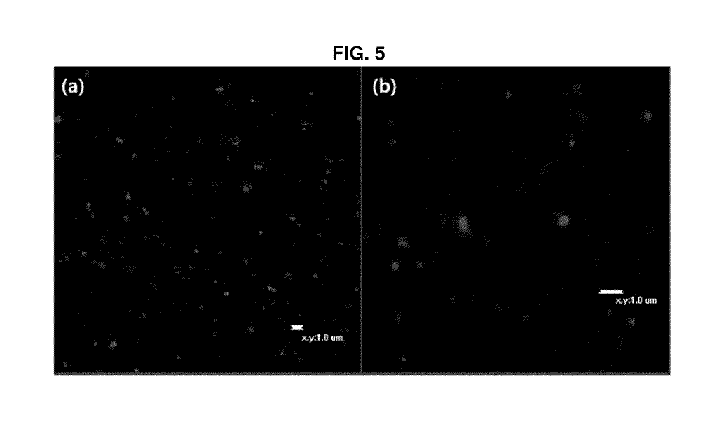

To measure a size of the artificial lipid-polymer-DNA complex formed by the method of Example 2, the artificial lipid-polymer-DNA complex whose size was homogenized using a particle homogenizer (extruder) was stained with rhodamine B by the same method as described in Example 3, and observed with a confocal microscope, an atomic force microscope and by laser scattering. The results are shown in FIGS. 5 to 7B, respectively.

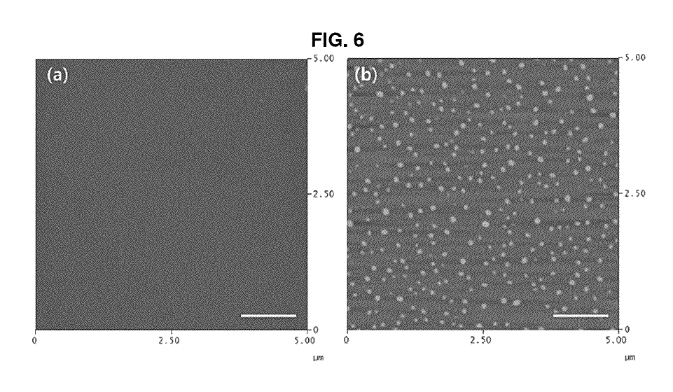

As shown in FIGS. 5(a), 5(b), 6(a) and 6(b), a size of the lipid membrane surface support including the polymer matrix was measured at approximately 200 nm when observed with a confocal microscope.

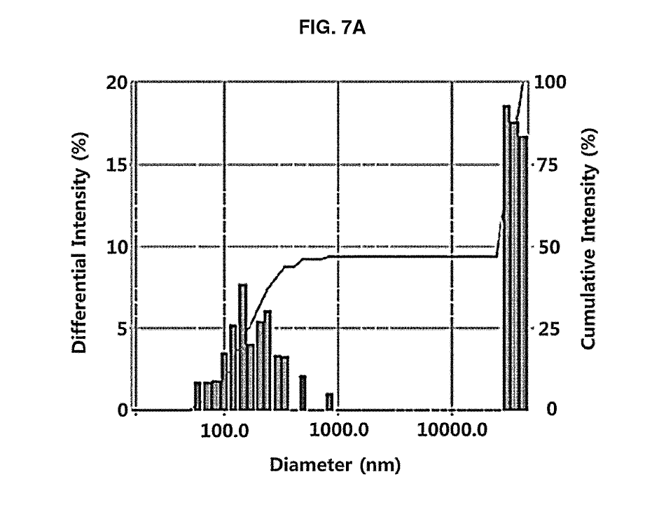

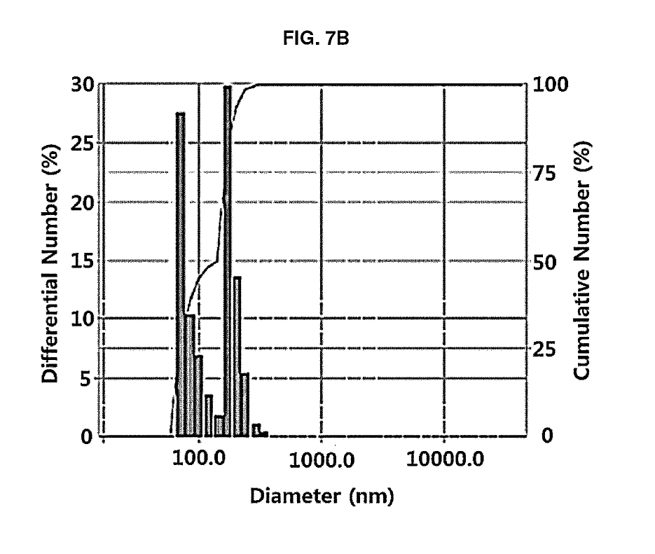

In addition, as shown in FIGS. 7A and 7B, it was confirmed that the result measured using laser scattering was 165.1±384.6 nm, and a size of the artificial lipid-polymer-DNA complex measured by the method of Example 2 was averagely 200 nm. According to the results, it was confirmed that the artificial lipid-polymer-DNA complex having a uniform size could be formed using a particle homogenizer (extruder).

### Example 5

### Confirmation of Structure of In-Vivo Imaging Agent

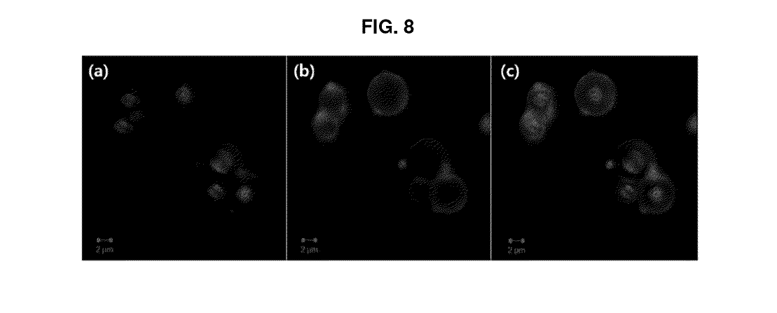

To confirm a structure of an artificial lipid-polymer-DNA complex including a fluorescent-labeled nucleic acid, that is, an in-vivo imaging agent, a lipid membrane surface support including a polymer matrix containing salmon sperm DNA formed by the method of Example 2 was stained with rhodamine B, and observed using a confocal microscope. The result is shown in FIG. 8.

As shown in FIG. 8(c), it could be confirmed that salmon sperm DNA stained with SYBR Green (green) was contained in the artificial lipid-polymer-DNA complex stained with the rhodamine B (red), and a size of the DNA-containing artificial lipid-polymer-DNA complex was approximately 2 μm.

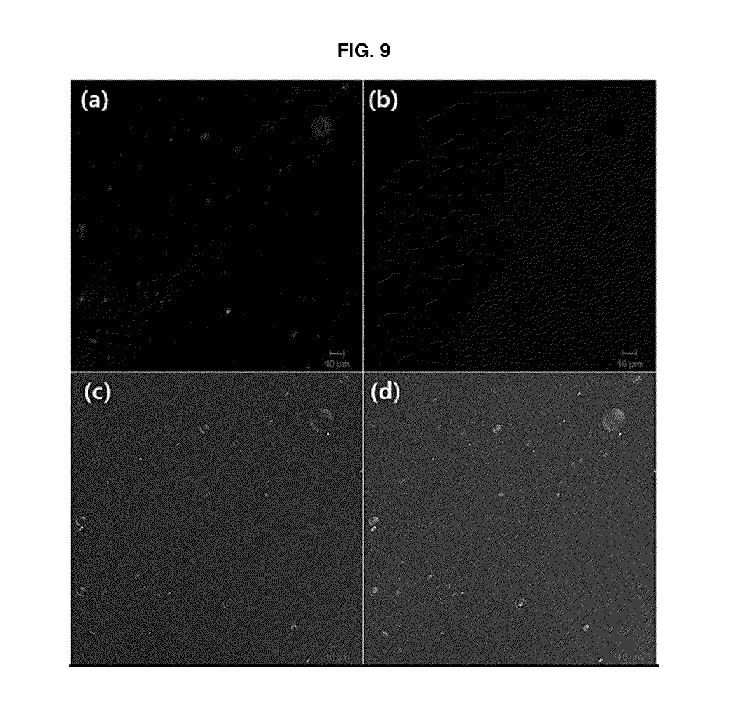

To confirm that the DNA was contained in the polymer matrix, a lipid was removed using Triton X-100 by the same method as described in Example 3, and stained with rhodamine B, and the resulting product was observed using a confocal microscope. The result is shown in FIG. 9.

Referring to FIG. 9(b), removal of the lipid by Triton X-100 was confirmed through the fact that the rhodamine B (red)-stained lipid was not observed. Referring to FIG. 9(d), it was confirmed that, after the lipid was removed, the salmon sperm DNA stained with SYBR Green (green) was observed in the polymer matrix by an optical means. According to the results, it was confirmed that the salmon sperm DNA was normally contained in the polymer matrix, and it was also confirmed that, in addition to the salmon sperm DNA, other DNAs were also stained with a fluorescent material, and normally contained in the artificial lipid-polymer-DNA complex.

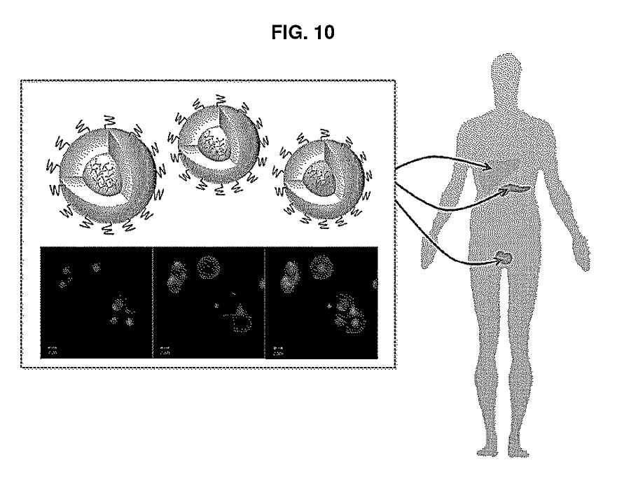

FIG. 10 illustrates an example of a method of providing information for diagnosing a disease using an in-vivo imaging agent according to the present disclosure. Referring to FIG. 10, an example of the in-vivo imaging agent may include a nucleic acid aptamer disposed on the outer surface of the lipid bilayer, which provides the in-vivo imaging agent with a specificity for a specific target tissue. An in-vivo imaging agent using a nucleic acid-based nano barcode can be stably introduced into the body of a living organism, delivered to a desired location with a great specificity, and have considerably low in-vivo toxicity and high in-vivo stability.

Described above are examples of a bioimaging agent using a nucleic acid-based nanobarcode; a bioimaging agent of a novel concept in which a fluorescent material-labelled nucleic acid is contained inside an artificial lipid-polymer-DNA complex comprising a polymer matrix composed of poly(D,L-lactide-co-glycolide) inside a surface support composed of a lipid membrane; a preparation method thereof; and a use thereof. The bioimaging agent using a nucleic acid-based nanobarcode may include a lipid membrane simulating a biomembrane so as to be stably introduced into cells in vivo, and can precisely transfer a nucleic acid-based nanobarcode to a target tissue by combining a target-specific nucleic acid aptamer to the surface of a lipid membrane, thereby remarkably increasing target-specific imaging efficiency. In addition, it is possible to remarkably lower toxicity in the body and to increase stability by forming a bilayer using biodegradable and biocompatible 50:50 poly; (D,L-lactide-co-glycolide). Further, since drugs for treating various diseases can be freely contained inside the bioimaging agent, the bioimaging agent is expected to be a fundamental technology capable of being applied to both bio-diagnosis and therapy.

While the disclosure has been shown and described with reference to certain exemplary embodiments thereof, it will be understood by those skilled in the art that various modifications in form and details may be made therein without departing from the spirit and scope of the disclosure as defined by the appended claims.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described composition, device, material or agent are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An artificial lipid-polymer-DNA complex, comprising:
a polymer matrix comprising polylactic-co-glycolic acid disposed within a lipid membrane.

2. The artificial lipid-polymer-DNA complex according to claim 1, wherein the lipid membrane comprises 1,2-dioleoyl-sn-Glycero-3-Phosphocholine, 1,2-dioleoyl-sn-glycero-3-[phospho-rac-(1-glycerol)]), and 1,2-dioleoyl-sn-glycero-3-phosphoethanolamin-N-[4-(p-maleimidophenyl)butyramide].

3. The artificial lipid-polymer-DNA complex according to claim 1, wherein the artificial lipid-polymer-DNA complex has a spherical shape with a diameter of 50 nm or greater and 10 μm or less.

4. The artificial lipid-polymer-DNA complex according to claim 1, wherein the artificial lipid-polymer-DNA complex is configured to deliver in vivo a therapeutic agent disposed in the polymer matrix to a tissue targeted by specificity determined by a nucleic acid aptamer disposed on a surface of the lipid membrane.

5. An in-vivo imaging agent, comprising:
a nucleic acid in the artificial lipid-polymer-DNA complex of claim 1.

6. The agent according to claim 5, wherein the artificial lipid-polymer-DNA complex has a nucleic acid aptamer having cancer cell targeting ability on its surface.

7. The agent according to claim 5, wherein the nucleic acid is labeled with a fluorescent material.

8. The agent according to claim 5, wherein the nucleic acid comprises a nucleic acid-based nano barcode having cancer cell targeting ability.

9. The agent according to claim 8, wherein the nucleic acid-based nano barcode is combined with at least one linear nucleic acid comprising one selected from the group consisting of the base sequences of SEQ. ID. NOs: 1 to 9.

10. The agent according to claim 5, wherein the in-vivo imaging agent includes an anticancer agent.

11. A pharmaceutical composition for diagnosing or treating a cancer, comprising:
the in-vivo imaging agent according to claim 5 as an active component.

12. A method of preparing an in-vivo imaging agent, comprising:
preparing a mixed solution by dissolving 1,2-dioleoyl-sn-glycero-3-phosphocholine1,2-dioleoyl-sn-glycero-3-[phospho-rac-(1-glycerol)], and 1,2-dioleoyl-sn-glycero-3-phosphoethanolamin-N-[4-(p-maleimidophenyl)butyramide] in a concentration ratio of 40:10:50 to 72:18:10 in an organic solvent;
preparing a lipid mixture by drying the mixed solution and dissolving the dried product in dichloromethane;
preparing a polymer-lipid mixed solution by adding a polylactic-co-glycolic acid (50:50 Poly(D,L-lactide-co-glycolide)) polymer solution dissolved in dichloromethane to the lipid mixture;
adding the polymer-lipid mixture to distilled water from which a DNase comprising a nucleic acid is removed, and performing sonication;
evaporating dichloromethane from the sonicated polymer-lipid mixture; and
homogenizing a particle size using a particle homogenizer (extruder).

13. The method according to claim 12, wherein the adding of the polymer-lipid mixture to distilled water from which a DNase comprising a nucleic acid is removed is performed at a temperature of greater than 2° C. and less than 8° C.

14. The method according to claim 12, further comprising:
binding a nucleic acid aptamer having cancer cell targeting ability to 1,2-dioleoyl-sn-glycero-3-phosphoethanolamin-N-[4-(p-maleimidophenyl)butyramide].

15. The method according to claim 12, wherein the organic solvent is chloroform or a chloroform-methanol mixed solution.
